(19) United States
(12) Patent Application Publication
(10) Pub. No.: US 2002/0025325 A1
(43) Pub. Date: Feb. 28, 2002

(54) Methods and composition for oral vaccination

(75) Inventors: Hsien-Jue (Steve) Chu, Fort Dodge, IA (US); Wumin Li, Fort Dodge, IA (US)

Correspondence Address: AMERICAN HOME PRODUCTS CORPORATION, FIVE GIRALDA FARMS, PATENT LAW, MADISON, NJ, 07940, US

(73) Assignee: American Home Products Corporatio, Madison, NJ

(21) Appl. No.: 09/887,296

(22) Filed: Jun. 21, 2001

Related U.S. Application Data
(60) Provisional application No. 60/215,359, filed on Jun. 30, 2000.

(51) Int. Cl. A61K 39/295; A61K 39/12; A61K 39/02

(52) U.S. Cl. 424/204.1; 424/201.1; 424/234.1

(57) ABSTRACT

The present invention encompasses methods and compositions both for providing protection against disease in an animal and for inducing increased intake of an orally administered vaccine by an animal. The methods of the invention are directed to admixing a bacterial or viral antigen with a water soluble palatable flavorant, further admixing the antigen and flavorant mixture with a water soluble vehicle for oral administration of the vaccine to an animal in order to provide protection against disease associated with infection by the admixed antigen and to induce the increased intake of the vaccine with the flavorant. The present invention thus encompasses methods and compositions for the oral vaccination of healthy animals through drinking water or syrups as an aid in the prevention of disease. The admixing of the palatable flavorant provides for a vaccine formulation with a desirable taste in order to promote self-administration of the vaccine formulation and/or to prevent rejection of the formulation when administered by an animal handler.

### FIELD OF THE INVENTION

The present invention is directed to methods and composition for the oral vaccination of healthy animals through drinking water or syrups as an aid in the prevention of disease.

### BACKGROUND OF THE INVENTION

There are a number of infectious diseases that can afflict populations of animals which cause weakening and death. Successful vaccination against such infectious diseases has previously been carried out in order to ameliorate or eliminate the symptoms of disease in infected animals. Orally administered vaccination is a preferable method as it removes the necessity for injection.

In large populations of farm animals, such as swine, poultry, cattle, sheep, goats and horse, vaccination by injection can be time consuming and labor intensive. In addition, intramuscular injection may cause damage to meat and stress to the animal.

In domesticated pets, such as dogs and cats, the stress of receiving an intramuscular injection would be alleviated by the use of an efficacious oral vaccine against common infections.

The size of both swine and poultry units has grown considerably throughout the world. Many swine facilities are now able to hold more than 10,000 weaned pigs, while many poultry units are now able to hold even more birds. Vaccination of each pig or bird with traditional vaccines is both labor intensive and difficult. Each animal must be captured, injected at least once, and in many cases twice, and accounted for during the vaccination process.

Because of these challenges, an efficacious vaccine administered to groups of animals through drinking water (mass administration) that would protect the swine or poultry from infection would be of great benefit to producers by saving labor costs as well as avoiding stress and damage to the meat caused by needles.

In addition producers of cows, sheep, goats and horses, which are generally raised in barns and are often housed or penned separately from one another, would also benefit from an oral vaccine administered through drinking water so as to relieve the costs of individual injection, stress and meat damage.

Finally, domesticated pets, such as dogs and cats, would benefit from administration of oral vaccines so as to reduce their stress and avoid injections.

Previously, the chief disadvantages experienced during mass administration of vaccine through drinking water to large groups of birds has been the inconsistency of vaccine dosage due to fluctuations in water consumption and the potential for some animals to receive no vaccine at all. In addition, viability and stability of the bacterial or viral agent in the vaccine can be affected upon admixing in water. Stability in water can decline dramatically over time. It would thus be highly desirable to provide a vaccine for mass administration to animals in a limited amount of time so as to prevent destabilization of the immunogenic agent. It would also be advantageous to provide a vaccine which is desirable to the animals in order to ensure consistent self administration of vaccine-containing drinking water throughout the population.

Another major drawback to oral administration of vaccines against disease causing infectious agents is that such agents are often associated with an unpleasant odor or taste. Vaccine formulations which are mass administered to large groups of animals must be desirable to the animals otherwise they will not self-administer them, i.e. drink them. In the same way, it would be advantageous for vaccine formulations administered to barn animals or animals that are individually penned to be palatable to the animals so that they self-administer the formulations. Finally, with regard to domesticated pets, these animals generally receive oral vaccines in the mouth which are administered by the veterinarian or animal health care worker and are often rejected by the animal and spit out. Thus, it would be highly advantageous to provide the orally administered vaccines in a formulation that would be desirable to the animal and increase the likelihood of successful administration and intake of the vaccine.

WO 98/51279 describes the administration of an oral vaccine comprising DNA encoding antigenic peptides which are incorporated into polymeric microparticles. Taste enhancing agents may be incorporated into the microparticles. However, such microparticles are not water soluble and do not provide for the administration of bacteria or viruses which cause disease.

Bell, et al. (Australian Veterinary Journal 68 (3), 1991, pp. 85-89) describe the administration of Newcastle disease V4 strain vaccine via mass administration to chickens The vaccine was administered utilizing the following three methods: 1) admixing with skim milk and administration in drinking water; 2) administration in an aerosol; and 3) administration in a coarse spray. While serological evidence of the generation of antibodies against Newcastle virus was demonstrated, no viral challenge studies were performed. It was thus not possible to determine the extent of vaccination against disease in these birds. More importantly, no attempts were made to make the vaccine formulation more palatable to the birds.

Grieve describes the evaluation of vaccines mass administered to chickens through drinking water or spray by the addition of a blue dye to a Newcastle disease vaccine vaccine formulation. The dye is used in order to monitor the consumption of the vaccine by the birds by temporarily staining the tongues of the birds. The dye demonstrated that only approximately 80% of the flock consumed the vaccine. No attempts were made to make the vaccine formulation more palatable to the birds.

It would thus be highly desirable to formulate and administer an efficacious labor-saving orally administered vaccine which is palatable to animals. Such vaccine formulations could offer veterinarians and milk and meat producers a convenient new strategic tool for optimizing herd and other animal health, while a more palatable oral vaccine which is not rejected by the animal would be desirable in veterinary practice.

### SUMMARY OF THE INVENTION

The present invention encompasses a method of providing protection against disease in an animal comprising:

(a) admixing a water soluble palatable flavorant with a water soluble vehicle for administration of an orally administered vaccine;

(b) further admixing with the mixture of step (a), an antigen selected from the group consisting of a bacterium and a virus as an active component of the orally administered vaccine; and

(c) administering the orally administered vaccine of step (b) to an animal to provide protection against disease associated with infection by the antigen.

The present invention also encompasses a method of inducing increased intake of an orally administered vaccine by an animal comprising:

(a) admixing a water soluble palatable flavorant with a water soluble vehicle for administration of an orally administered vaccine;

(b) further admixing with the mixture of step (a), an antigen selected from the group consisting of a bacterium and a virus as an active component of the orally administered vaccine; and

(c) administering the vaccine admixture of step (b) orally to the animal;

(d) inducing the increased intake of the orally administered vaccine with the flavorant.

The present invention further encompasses an orally administered animal vaccine formulation comprising as an active component an antigen selected from the group consisting of a bacterium and a virus, a water soluble palatable flavorant and a water soluble vehicle for administration of the orally administered animal vaccine.

### DETAILED DESCRIPTION OF THE INVENTION

All patents, patent applications, publications and other materials cited herein are hereby incorporated by reference in their entirety. In the case of inconsistencies, the present description, including definitions, is intended to control.

As used herein, the term “mass administration” is defined as the large scale administration of water soluble vaccine to groups of animals that are held together in large facilities. Typically, such facilities house swine and poultry.

As used herein, the terms “swine” and “pig” or “pigs” are used synonomously.

As used herein, the term “poultry” is defined as including chickens, turkeys and ducks.

As used herein, the term “palatable flavorant” is defined as a taste enhancing agent which is demonstrated to be desired by the animal or animals to which it is administered. Such desirability is determined prior to formulation into the orally administered vaccine of the invention through observation of self administration of drinking water or syrup which have been flavored with the palatable flavorant. Non-limiting examples of such flavorants include fruit flavors such as strawberry, cherry, grape, watermelon, apple and the like; fish flavors; meat flavors; and any other flavorants that are preferred by the animal or animals. Fruit flavorants are particularly preferred for administration to pigs, horses, sheep, goats, cats and dogs. Meat flavorants are particularly preferred for dogs and cats. Fish flavorants are particularly preferred for cats.

The term “animal handler” as used herein includes a farm worker, veterinarian, animal health professional or other person responsible for the care of the animal and administration of medicines, vaccines and/or foods to the animal.

The present invention encompasses methods and compositions both for providing protection against disease in an animal and for inducing increased intake of an orally administered vaccine by an animal. The methods of the invention are directed to admixing a bacterial or viral antigen with a water soluble palatable flavorant, further admixing the antigen and flavorant mixture with a water soluble vehicle for oral administration of the vaccine to an animal in order to provide protection against disease associated with infection by the admixed antigen and to induce the increased intake of the vaccine with the flavorant.

The present invention thus encompasses methods and compositions for the oral vaccination of healthy animals through drinking water or syrups as an aid in the prevention of disease. The admixing of the palatable flavorant provides for a vaccine formulation with a desirable taste in order to promote self-administration of the vaccine formulation and/or to prevent rejection of the formulation when administered by an animal handler.

The antigens formulated into the vaccines of the invention are bacterial and viral disease causing agents. Live bacteria and viruses are particularly preferred. When administering live bacteria or virus as the antigen in a vaccine formulation, the viability of the live antigen is of particular concern. The animal or animals must take in the vaccine before the viability of the antigen is greatly diminished so as to ensure the greatest possible antigenicity and to obtain a strong immune response.

An “avirulent” or “inactivated” bacterial or viral strain is understood to be one that is not able to cause disease in an animal and includes any strain that a person of skill in the art would consider safe for administering to an animal as a vaccine. For example, a strain causing minor clinical signs, which may include fever, serous nasal discharge or ocular discharge, is within the scope of the present invention since such clinical signs are considered acceptable vaccine side effects.

One method of inactivating bacterial or viral antigens for use in the invention is to introduce gene mutations such as nucleotide substitutions, insertions and/or deletions in the genome of the antigen which abrogate its ability to cause disease. Methods of recombinant DNA technology can be used to engineer deletions, insertions and substitutions in the bacterial or viral antigen genome to produce attenuated strains. These methods are well known in the art and are described, for example, in Sambrook et al. (Molecular Cloning, A Laboratory Manual, 2nd ed., Cold Spring Harbor Laboratory Press, 1989). Other methods of attenuating or inactivating a bacterial or viral antigen for use in the invention are well known to those of ordinary skill in the art.

As used herein, a “modified live virus” or “modified live bacteria” is a viral or bacterial antigen that has been altered, typically by passaging in tissue culture cells, to attenuate its ability to cause disease, but which maintains its ability to protect against disease or infection when administered to animals.

An “infectious unit” of a viral antigen of the invention is defined as a TCID₅₀, or the amount of virus required for infecting or killing 50% of tissue culture cells.

The concentration of bacterial antigen in a given culture can be determined by standard methods known in the art, such as, for example, microscopic analysis, colony count or spectrophotometric analysis of a liquid culture.

The concentration of a bacterial toxin antigens can be obtained by determining the lethal dose (LD) and LD₅₀ in a suitable animal model, e.g., mouse.

The vaccine may be prepared from freshly harvested viral cultures by methods that are standard in the art. The growth of the virus is monitored by standard techniques (observation of cytopathic effect, immunofluorescence or other antibody-based assays), and harvested when a sufficiently high viral titer has been achieved. The viral stocks may be further concentrated or lyophilized by conventional methods before inclusion in the vaccine formulation. Other methods, such as those in described in Thomas, et al., Agri-Practice, V.7 No. 5, pp.26-30., can be employed.

Bacteria are grown according to known methods in the art. The bacterial antigens to be used in the formulations of the invention may liquid form or may also be of a lyophilized form to be reconstituted prior to use with the palatable flavorant and water soluble vehicle.

Generally, the preferred amount of a bacterial antigen to be administered in a dose of vaccine for a single animal is from about 10⁵ to about 10¹¹ Colony Forming Units (“CFU”), preferably from about 10⁶ to about 10¹⁰ CFU, and most preferably from about 10⁷ to about 10⁹ CFU. In another preferred embodiment, the effective amount is from about 10⁵ to about 10⁸ CFU per dose.

Generally the preferred amount of a viral antigen to be administered in a dose of vaccine for a single animal should contain an amount corresponding to from about 10^{3.0} to about 10^{6.0} TCID₅₀/ml, preferably 10⁴ to 10⁵ TCID₅₀/ml.

The dosage or effective amount for each particular bacterial or viral antigen to be formulated into the vaccines of the invention will generally depend on the age, health and immune status (e.g., previous exposure, maternal antibody) of the animal or animals being vaccinated, as well as the particular antigen being used. A suitable effective amount, including the minimum antigen level and water or syrup dosage calculation to be administered can be routinely determined by those of ordinary skill in the art.

As noted above, any infectious, attenuated or inactivated, live or dead bacterial or viral agent may be formulated into the vaccines of the invention and administered according to the methods of the invention. Non-limiting examples of particularly preferred antigens include those that infect the following animals:

Swine—Erysipelothrix rhusiopathiae, Actinobacillus pleuroneumonla, Mycoplasma hyopneumonlae, E. coli K88, K99, F41 and 987P, Clostridium perferingens type c, Salmonella choleraesuls, Pasterurella muitocida, Bordetella bronchiseptica, Leptospira bratislava, Leptospira canicola, Leptospira grippotyphosa, Leptospira hardjo, Leptospira promona, Leptospira ictero, Porcine Influenza virus, Circovirus, PRRS virus, Swine pox, Rotavirus, Porcine Respiratory Coronavirus, Parvo virus, Pseudorabies, transmissible gastroenteritis agent.

Horses—Streptococcus equi, Clostridium tetani, Equine Influenza Virus A1 and A2 strains, Equine Rhinopneumonids type 1, 1b and 4, Eastern Equine Encephalomyelitis, Western Equine Encephalomyelitis, Venezuelan Equine Encephalomyelitis, Equine Rotavirus.

Cattle—E. coli O157:H7, Pasterurella multocida, Pasterurella haemolytica, Leptospira canicola, Leptospira grippotyphosa, Leptospira hardjo, Leptospira promona, Leptospira Ictero, Clostridium perferingens type C, Clostridium perferingens type D, Clostridium chauvoel, Clostridium novyl, Clostridium septicum, Clostridium tetanus, Clostridium haemolyticum, Clostridium sodellii, Salmonella dublin and typhimurium, Bovine Rotavirus, Bovine coronavirus, Bovine rhinotracheitis, Bovine diarrhea virus, Parainfluenza-3, Respiratory syncytial virus.

Poultry—Salmonella typhimurium, Sepullina pilosicoli, Marek's disease virus, Infectious bursal disease, Infectious bronchitis, Newcastle disease virus, Reo virus, Turkey rhinotracheltis, Couidiosis.

Dog—Leptospira canicola, Leptospira grippotyphosa, Leptospira hardjo, Leptospira promona, Leptospira ictero, Canine Borrella burgdorferi, Canine Ehrlichia canis, Canine Bordetella bronchiseptica, Canine Giardia lambia, Canine distemper, Canine Adenovirus, Canine Coronavirus, Canine Parainfluenza, Canine Parvovirus, Canine Rabies.

Cat—Feline Chlamydia psittaci, Feline immunodeficiency virus, Feline infectious peritonitis virus, Feline leukemia virus, Feline rhinotrachelitis, Feline Panleukopenia, Feline rabies. In many instances the preparation and production of the bacterial and viral antigens for formulation into the orally administered vaccines of the invention results in an antigen with an unpalatable taste that the animals do not like. Thus, when orally administering the vaccine either in drinking water or a syrup, the animals will either not drink as much of the vaccine formulation or will reject the syrup and spit it out due to an unpleasant taste. The admixing of a palatable flavorant into the vaccine formulations of the invention promotes and increases the intake of the orally administered vaccines. Such palatable flavorants are admixed at a concentration dictated by the flavorant utilized. Preferred concentrations include at least about 0.01% to 1.0% or more.

Liquid flavorants may be added to the vaccine formulations by dropper or other means. If the flavorants are in powdered form, they may be rehydrated and mixed into the vaccine formulation.

When administering the oral vaccines of the invention to pigs or poultry, the preferred method of administration is through mass administration to large groups of animals that are housed together. The vaccine is formulated into drinking water that is provided to the animals through a continuous feed or drip with the animals then going to the drinking water and self administering the vaccine by drinking the vaccine contained in the water. One example of a continuous feed or drip device is an automated water proportioning device called a Dosatron™ (Dosatron International Inc., Clearwater, Fla.) In a preferred embodiment, the water proportioning device provides a continuous feed of the water soluble vaccine/flavorant in small amounts to a water drip feeder that then provides water to the animals through mass admionistration into the housing facility, such as by dripping through nipples.

When administering the oral vaccines of the invention to cattle, horses, sheep, goats or other farm animals which are permanently housed or maintained separately in a barn, stall, or pen, the preferred method of administration is through administration in a bucket or trough of drinking water.

When administering the oral vaccines of the invention singly to an animal or a to domesticated pet such as a cat or dog, the vaccine may be administered in drinking water or, more preferably, in a syrup. Such syrup is preferably administered into the mouth through a device such as a syringe. Such administration is most preferably at the back of the throat. The oral vaccines may be formulated into a syrup according to known methods in the art. Non-limiting examples of methods of formulating syrups can be found in the following references:

“Preparation of high conversion syrups by using thermostable amylases from thermoanaerobes”, Saha, B. C.; Zeikus, J. G., Enzyme And Microbial Technology, Vol.12, No.3, p.229-231 (1990);

“Problem of The Mass-Volume Preparation of Medicinal And Table Syrups”, Bondarenko, A. I., Farmatsiya (Moscow), Vol.33, No. 6, p.70-71 (1984);

“Pharmaceutical development of a new syrup formulation versus cough: From test-size batch to pilot-size batch.”, Renaudeau, P.; Clair, P.; Caire-Maurisier, F., Travaux Scientifiques des Chercheurs du Service de Sante des Armees, Vol. 0., No. 20. (1999), pp. 113-114;

“Formulation and evaluation of sustained-release dextromethorphan resinate syrup”, El-Samaligy, M. S.; Mahmoud, H. A.; Omar, I. M., Egyptian Journal of Pharmaceutical Sciences, Vol. 37, No. 1-6 (1996), pp. 509-519;

“Pharmacokinetics, efficacy, tolerance of a new formulation of quinine (syrup) in uncomplicated malaria in children.”, Rey, E.; Pariente-Khayat, A.; D'Athis, P.; Tetanye, E.; Varlan, M.; Olive, G.; Pons, G., Methods and Findings in Experimental and Clinical Pharmacology, Vol. 18, No. Suppl. B (1996), pp. 125;

“Therapeutic bioequivalence between drop and syrup formulations of a (dextromethorphan-guaifenesin-menglithate)-based cough suppressant.”, Franchi, F., Rivista di Patologia e Clinica, Vol. 48, No. 3 (1993), pp. 149-166;

“Continuous preparation of fructose syrups from Jerusalem artichoke tuber using immobilized intracellular inulinase from Kluyveromyces sp. Y-85”, Wei,

Wenling; Le Huiying, Wan Wuguang; Wang, Shiyuan, Process Biochem. (Oxford), Vol. 34, No. 6, 7 (1999), pp. 643-646;

“Syrups for preparation of impact-modified polymers with large particle size”, Doyle, Thomas R., Oct. 26, 1999, U.S. Pat. No. 5,973,079;

“Enzymatic preparation of glucose syrup from starch”, Norman, Barrie Edmund; Hendriksen, Hanne Vang, Sep. 16, 1999, WO 99/46399;

“Acrylate syrup composition with good weather resistance”, Makino, Takayuki; Takemoto, Toshio; Yanagase, Akira, Aug. 3, 1999, Japanese Patent No. 99209431 (Japanese Patent Application No. 1998-24041-A2);

“Microelement syrup and method of its preparation”, Sviatko, Peter; Boda, Koloman, Jul. 8, 1998, Slovakian Patent No. 279,128;

“Monitoring beet sugar evaporator syrup invert and sucrose composition by ion chromatography”, Vercellotti, John R.; Desimone, Frank; Clarke, Margaret A., Proc. Sugar Process. Res. Conf. (1998), pp. 442-448;

“Preparation of powders from trehalose syrups”, Totsuka, Atsushi; Yamamoto, Takeshi; Umino, Takehiro, May 25, 1999, Japanese Patent No. 99140094 (Japanese Patent Application No. 1997-315993/A2 filed Oct. 31, 1997);

“Human IGF-I syrup composition and its use”, Shirley, Bret A.; Hora, Maninder S., May 20, 1999, WO 99/24062;

“The effect of carbohydrate composition of starch syrups on the quality and the stability of foam products”, Nebesny, Ewa; Pierzgalski, Tadeusz; Rosicka, Justyna, Zesz. Nauk.—Politech. Lodz., Chem. Spozyw. Biotechnol., Vol. 58 (1998), pp. 69-94;

“Preparation of chloral hydrate syrup”, Ishida, Atsuyo; Miyama, Shuho; Mikayama, Hiroki; Teruyama, Shigeo; Takeyasu, Akiko; Ohasi, Atsushi; Okamoto, Kazuaki; Onishi, Toshio; Yasuhara, Akihiro, Igaku to Yakugaku, Vol. 40, No. 2 (1998), pp. 329-333;

“Properties and composition of concentrates and syrup obtained by microfiltration of saccharified corn starch hydrolyzate”, Singh, N.; Cheryan, M., J. Cereal Sci., Vol. 27No. 3 (1998), pp. 315-320;

“Process for the preparation of crystalline lactulose from commercial syrups”, Bimbi, Giuseppe, European Patent No. 622,374-B1;

“Maltitol based sweetening syrup, confections produced using this syrup and the use of a crystalization propagation controlling agent in the preparation of these products”, Ribadeau-Dumas, Guillaume; Fouache, Catherine; Serpelloni, Michel, European Patent No. 611,527-B1;

“Syrup composition”, Kawasaki, Yoshihiko; Suzuki, Yukio, European Patent No. 441,307-B1;

“Carbohydrate Syrups and Methods of Preparation”, PATEL, Mansukh, M.; REED, Michael, A.; WOKAS, William, J.; KURES, Vasek, J.; European Patent No. 241,543-B1;

“Methadone syrup formulation for diabetic heroin drug addict patients”, Gagnaire, L.; Fellous, J.; Dauphin, A.; Bonan, B., Journal de Pharmacie Clinique (France), Vol. 17, No. 4 (1998), pp. 264-267;

“Application of solubilizers on the preparation of stable syrups containing Extractum plantaginis fluidum”, Tichy, E., Pharmazie (Germany), Vol. 52, February 1997, pp. 167-168;

“Double-blind, placebo-controlled, pharmacokinetic and -dynamic studies with 2 new formulations of piracetam (infusion and syrup ) under hypoxia in man”, Saletu, B.; Hitzenberger, G.; Grunberger, J.; Anderer, P.; Rameis, H. et al., International Journal of Clinical Pharmacology and Therapeutics, Vol. 33, May 1995, pp. 249-262;

“Bioavailability of syrup and tablet formulations of cefetamet pivoxil”, Ducharme, M. P.; Edwards, D. J.; McNamara, P. J.; Stoeckel, K., Antimicrobial Agents and Chemotherapy, Vol. 37, December 1993, pp. 2706-2709;

“Comparison of sprinkle versus syrup formulations of valproate for bioavailability, tolerance, and preference”, Cloyd, J. C.; Kriel, R. L.; Jones-Saete, C. M.; Ong, B. Y.; Remmel, R. P. et al., Journal of Pediatrics, Vol. 120, April 1992, pp. 634-638;

“Preparation of syrups rich in fructose from tupinambo”, Magro, J. Regalo Da; Fonseca, M. M., Revista Portuguesa de Farmacia (Portugal), Vol. 38, Apr.-Jun. 1988, pp. 27-32;

“The clinical study of cefpodoxime proxetil dry syrup preparation in the pediatric field”, Kasagi, T.; Tanimoto, K.; Ogihara, Y.; Hayashibara, H.; Okuda, H.; Shiraki, K., Jpn J Antibiot, Vol. 47, No. 9, September 1994, pp. 1202-9; and

“Acetaminophen or phenobarbital syrup composition”, Kawasaki, Yoshihiko; Suzuki, Yukio, U.S. Pat. No. 5,154,926.

The amount of vaccine stock solution prepared is based on the amount of water each animal would drink during the vaccination period. Preferred vaccination periods are from 0.5 to 10 hours for administration in drinking water depending on the antigen. The amount of water each animal would drink is estimated according to the average body weight of the animals to be vaccinated. When using a automated water proportioning device, a preferred method is as follows: The vaccine stock solution is added to the automated water proportioning device via a connecting hose, which is in turn connected to the water source. The water proportioning device pumps the vaccine along with running water into the pipeline and toward the nipple or nipples through which the drinking water drips.

To formulate the orally administered vaccines of the invention, an initial determination of the quantity of water (based on body weight) to be administered to the animals is made. The total weight of the animal(s) to be vaccinated is determined by calculating the total number of animals to be vaccinated multiplied by the average weight of the animal. The quantity of water needed for the weight of animal(s) is determined and the vaccine formulation is caluclated based on the required water and time span over which the vaccine formulation is to be administered. One non-limiting example of the types of calculation methods to be used in the formulation and administration of the vaccines of the invention to pigs can be found in Example 1 and Table 2.

The average quantity of water to be administered to the animals of the invention can be determined by those of ordinary skill in the art. Non-limiting examples of the average quantity of water administered to: 1) poultry is from about 2.5-5 gallons per 1000 birds; 2) range cows consume a minimum of 2.5 gal. (9.5 L) of water/head/day in winter and up to 12 gal. (45 L)/head/day in summer; 3) breeding cows, yearlings, and 2-yr-old steers consume approximately 10 gal. (38 L) of water daily; 4) finishing calves drink 6-8 gal. (23-30 L) of water daily; and 5) small animals such as dogs and cats require approximately 250-1500 mL of water per day.

Prior to administration of the vaccine of the invention in drinking water, it is preferable to remove all drinking water from the animals to be vaccinated so as to promote intake of the drinking water. It is preferable to remove drinking water overnight prior to administration of the vaccine in drinking water.

The oral vaccines of the invention may be administered to the animals being immunized in a single dose or in two doses. A preferred method of the invention is the administration of two doses of the vaccine.

The following examples are intended as non-limiting illustrations of the present invention.

### EXAMPLE 1

Mass Administration of Oral Vaccine to Pigs Via Flavored Drinking Water

An immunogenicity study was conducted using a total of thirty 6 weeks of age pigs. Among the thirty pigs, twenty were vaccinates and ten were non-vaccinated controls. All twenty vaccinated pigs were mass vaccinated with Erysipelothrix Rhusiopathiae vaccine, Avirulent Live Culture, through drinking water using an automated water proportioning device (Dosatron). The second vaccination was given two weeks post first vaccination by using the same application method as the first one. All vaccinated pigs were observed for clinical signs associated with erysipelas eight days post each vaccination to ensure safety of the vaccine. Twenty-one days post second vaccination, all twenty vaccinates and ten non-vaccinated controls were challenged intramuscularly with a virulent strain of Erysipelothrix rhusiopathiae. All challenged pigs were observed through seven days post challenge for temperature and clinical signs associated with erysipelas in accordance with 9 CFR 113.67. None of the vaccinated pigs showed any clinical signs of erysipelas following each vaccination. After challenge, one hundred percent (100%) of the non-vaccinated control pigs showed severe clinical signs of erysipelas, including high temperature, arthritis, inappetence, depression, lethargy, generalized patchy redness (diamond-skin lesions) and sudden death during the observation period. Seventy percent (70%) of the control pigs were dead by 4-6 days post challenge. E. rhusiopathiae was isolated from all of the samples collected from the control pigs post challenge or at necropsy. In contrast, 100% of the vaccinated pigs did not show any clinical signs of erysipelas. Results from this study satisfactorily meet the requirements stated in 9 CFR 113.67 for an Erysipelothrix Rhusiopathiae Vaccine. Data collected from this study demonstrated that the mass vaccinated Erysipelothrix Rhusiopathiae Vaccine, Avirulent Live Culture, administered through drinking water, is safe and efficacious in protecting pigs from disease caused by E. rhusiopathiae at a minimum level of approximately 6.06×10⁷ CFU per dose.

TEST ANIMALS

| | |
|---|---|
| Species | Porcine |
| Number | 30 |
| Age | 6 weeks of age |
| Sex | Both |
| Breed | Mixed |
| Identification | Ear tag |
| Source | From FDAH SPF herd |

Housing and Care of Animals

All pigs were maintained on the sow until weaning at twenty-one days of age as is standard for the facility. Weaned pigs were given water and feed ad libitum. Pigs were started on antibiotic-free Early Start Feed (Supersweet Brand), and changed to Start Amino, as deemed appropriate by the site supervisor. The vaccinates and controls were housed in two separate rooms after vaccination until challenge. For administration of the vaccine: twenty vaccinated pigs were put into two pens with ten pigs per pen. Each pen was provided a water nipple connected to a water hose. Water to both nipples was driven by the same automated water proportioning device (Dosatron). At two days prior to challenge, the vaccinated pigs and non-vaccinated controls were commingled into one room and all the pigs were challenged with a virulent strain of E. rhusiopathiae. All challenged pigs remained in the room until the end of the observation period.

Composition of Vaccine

The lyophilized Erysipelothrix rhusiopathiae antigen used in this study was produced at the highest passage level (i.e.,Master Seed+5). The Master Seed of the antigen is cultures five times. Each passage is designated consecutively as MS+1, MS+2, MS+3, MS+4 and MS+5.

Experimental Design

Pigs were randomly assigned into vaccinate and control groups using a random number generator in Microsoft Excel. There were twenty vaccinates and ten non-vaccinated controls at 6 weeks of age at the time of first vaccination (Appendix 2). All vaccinates received two vaccinations at two weeks between doses. Both vaccinates and non-vaccinated controls were challenged at twenty-one days post second vaccination (21DPV2). For both vaccinations, the vaccine was delivered through drinking water using an automated water proportioning device (Dosatron). Serum samples from both vaccinates and controls were collected at the day of vaccination and the day of challenge for possible serological analysis in the future. Seven days post challenge (7DPC), all survived pigs were euthanized. Blood samples and organs were collected from control pigs post challenge or at necropsy for E. rhusiopathiae isolation. Blood samples were also collected from vaccinates at enthanization for E. rhusiopathiae isolation.

EVENT LOG

| Procedures | Age of Pigs |
|---|---|
| First vaccination | 6 weeks |
| Second vaccination | 8 weeks |
| Challenge | 11 weeks |
| Euthanization | 12 weeks |

APPENDIX 2
Body Weight of the Pigs Used in This Study

| | | Age | Body Weight | Body Weight |
|---|---|---|---|---|
| | | at First | at First | at Second |
| | | Vaccination | Vaccination | Vaccination |
| Group | Pig ID | (Day old) | (lb.) | (lb.) |
| Control | O403 | 38 | 17.6 | 36.1 |
| Control | O404 | 38 | 13.0 | 31.5 |
| Control | O406 | 38 | 19.1 | 37.0 |
| Control | O411 | 42 | 22.0 | 44.4 |
| Control | O417 | 42 | 18.0 | 37.0 |
| Control | O421 | 42 | 17.8 | 36.3 |
| Control | O426 | 41 | 18.3 | 33.9 |
| Control | O429 | 41 | 20.5 | 42.2 |
| Control | O432 | 41 | 12.1 | 30.1 |
| Control | R73 | 42 | 16.5 | 36.3 |
| Vaccinate | O401 | 38 | 17.2 | 35.9 |
| Vaccinate | O402 | 38 | 14.1 | 32.1 |
| Vaccinate | O405 | 38 | 14.1 | 31.2 |
| Vaccinate | O407 | 38 | 14.1 | 31.2 |
| Vaccinate | O409 | 42 | 26.0 | 45.1 |
| Vaccinate | O410 | 42 | 18.5 | 36.5 |
| Vaccinate | O412 | 42 | 23.1 | 35.0 |
| Vaccinate | O413 | 42 | 29.3 | 48.4 |
| Vaccinate | O414 | 42 | 11.0 | 27.9 |
| Vaccinate | O416 | 42 | 22.7 | 43.6 |
| Vaccinate | O419 | 42 | 16.7 | 33.0 |
| Vaccinate | O420 | 42 | 22.7 | 41.8 |
| Vaccinate | O422 | 42 | 12.5 | 26.0 |
| Vaccinate | O424 | 41 | 16.3 | 30.4 |
| Vaccinate | O425 | 41 | 21.8 | 40.7 |
| Vaccinate | O427 | 41 | 20.2 | 34.5 |
| Vaccinate | O428 | 41 | 17.2 | 35.9 |
| Vaccinate | O430 | 41 | 19.1 | 36.5 |
| Vaccinate | O431 | 41 | 17.2 | 38.1 |
| Vaccinate | R493 | 38 | 11.2 | 26.6 |
| Average of | | | 18.3 | 35.5 |
| Vaccinated | | | | |
| Pigs | | | | |

Preparation of Vaccine

The amount of vaccine stock solution prepared was based on the amount of water each pig would drink during the six hour vaccination period. The amount of water and vaccine organism each pig would drink was estimated according to the average body weight of the twenty pigs to be vaccinated (Appendix 3). Briefly, lyophilized vaccine was re-suspended in flavored (0.5% Givaudan Roure, Serial No.C-321110) diluent. The rehydrated vaccine was added to 5 liters of milk solution containing non-fat dry milk, and mixed well. The vaccine stock solution was further diluted to 7 liters using water and then the container was placed on a stir plate for further mixing. This stock solution was then connected to the automated water proportioning device via a connecting hose, which was in turn connected to the water source.

Appendix 3: Calculation of Estimated Amount of Vaccine Consumed During Vaccination Period

First Vaccination

1. Average body weight of vaccinates was 18.3 lb.

2. 18.3 lb/100 lb×946 mL=173 mL. This calculation was based on the assumption that a 100 lb pig would drink 1 gallon (3785.4 mL) of water during 24 hours, therefore, a 100 lb pig would drink 946 mL of water during 6 hour vaccination period.

3. Each vaccine bottle contained 4.12×10¹⁰ CFU (2.06×10⁹ CFU/mL×20 mL).

4. The targeted CFU per dose from nipples was 1×10⁸ CFU excluding the loss from the stock solution container to nipples.

5. In order for each pig to get 1×10⁸ CFU in 173 mL, the concentration of vaccine organism from nipples had to be 5.8×10⁵ CFU/mL (1×10⁸ CFU/173 mL)

6. To get 5.8×10⁵ CFU/mL from nipples, the concentration of vaccine stock solution had to be 7.42×10⁷ CFU/mL (5.8×10⁵ CFU/mL×128*=7.42×10⁷ CFU/mL).

7. To ensure the vaccine continually flowed out of the nipples during the 6 hour vaccination period, 7 liters of stock solution was needed. The total CFU in stock solution was 7.42×10⁷ CFU/mL×7000 mL=5.19×10¹¹ CFU.

8. Thirteen (13) bottles of the lyophilized vaccine were rehydrated with diluent, the amount of rehydrated vaccine that was equivalent to 12.6 bottles (5.19×10¹¹ CFU/4.12×10¹⁰ CFU/bottle=12.6 bottles) was mixed with non-fat milk and water to make the stock solution.

Second Vaccination

1. Average body weight of vaccinates was 35.5 lb.

2. 35.5 lb/100 lb×946 mL=336 mL. This calculation was based on the assumption that a 100 lb pig would drink 1 gallon (3785.4 mL) of water during 24 hours, therefore, a 100 lb. pig would drink 946 mL of water during 6 hour vaccination period.

3. Each vaccine bottle contained 4.12×10¹⁰ CFU (2.06×10⁹ CFU/mL×20 mL).

4. The targeted CFU per dose from nipples was 1×10⁸ CFU excluding the loss from stock solution container to nipples.

5. In order for each pig to get 1×10⁸ CFU in 336 mL, the concentration of vaccine organism from nipples had to be 2.98×10⁵ CFU/mL (1×10⁸ CFU/336 mL)

6. To get 2.98×10⁵ CFU/mL from nipples, the vaccine stock solution had to be 3.81×10⁷ CFU/mL (2.98×10⁵ CFU/mL×128*=3.81×10⁷ CFU/mL).

7. To ensure the vaccine continually flow out of nipples during the 6 hour vaccination period, 7 liters of stock solution was needed. The total CFU in stock solution was 3.81×10⁷ CFU/mL×7000 mL=2.67×10¹¹ CFU.

8. Seven (7) bottles of the lyophilized vaccine were rehydrated with diluent, the amount of rehydrated vaccine that was equivalent to 6.47 bottles (2.67×10¹¹ CFU/4.12×10¹⁰ CFU/bottle=6.47 bottles) was mixed with non-fat milk and water to make the stock solution.

*The proportioner was adjusted at 1:128 delivery ratio.

Preparation of Water System, Orally Administered Vaccine and Vaccination Procedure

The body weight of each vaccinated pig was measured on the day before vaccination (Appendix 2) and was used to calculate the amount of vaccine stock to be used during the vaccination period. Drinking water was withdrawn from the pigs overnight (at least 8-10 hours) prior to vaccination and re-delivered to the pigs after vaccination started. The vaccination period lasted six hours to ensure that the pigs consumed the estimated amount of vaccine. At the time of first vaccination, seven liters of stock vaccine were prepared as described above to ensure there was sufficient vaccine to continually flow out of the nipples during the six hour period. The Dosatron was connected to the stock solution container and the water proportioner was adjusted to deliver one ounce per gallon of water to the vaccinated pigs. The automated water proportioning device drove two water nipples (one nipple per pen) in parallel and delivered the vaccine to the two nipples simultaneously. The vaccine stock was placed on a stir plate to mix during the vaccination period. Samples from the two nipples were collected each hour after the delivery was started. Bacterial viable count was performed on TSA II agar plates with 5% sheep blood. Five plates were used for each sample.

At the time of second vaccination, the vaccine rehydration procedure, water proportioner set-up and sample collection were the same as for the first vaccination.

Calculation of Vaccination Dose

The concentration of vaccine and dose determination in the drinking water are shown in Appendix 4. The average viable count of the two nipples at first vaccination was 3.50×10⁵ CFU/mL and the estimated amount of water each pig consumed was about 173 mL, based on the group's body weight and the published water consumption rates. Therefore, the CFU per dose that each pig was actually administered was calculated to be 3.50×10⁵ CFU/mL×173 mL=6.06×10⁷ CFU.

Likewise, the average viable count of the two nipples at second vaccination was 1.42×10⁵ CFU/mL and the amount of water each pig would consume was about 336 mL. Therefore, the CFU per dose that each pig was actually administered during the second vaccination was calculated to be 1.42×10⁵ CFU/mL×336 mL=4.77×10⁷ CFU.

APPENDIX 4
Confirmation of Vaccine Viability and
Dose Determination in the Drinking Water

| | Sample | | |
|---|---|---|---|
| | Collected | | |
| | Post Initial | | |
| | Vaccina- | | |
| Vaccina- | ation | Nipple 1 | Nipple 2 |
| tion | Time (Hour) | (CFU/mL) | (CFU/mL) |
| First | 0 | 2.81E+05 | 2.68E+05 |
| First | 1 | 3.86E+05 | 2.86E+05 |
| First | 2 | 2.91E+05 | 3.48E+05 |
| First | 3 | 3.57E+05 | 3.71E+05 |
| First | 4 | 4.45E+05 | 4.24E+05 |
| First | 5 | 4.73E+05 | 4.34E+05 |
| First | 6 | 2.74E+05 | 2.54E+05 |
| First | Average | 3.58E+05 | 3.41E+05 |
| | | Average of | 3.50 × 10ˆ 5 |
| | | Two Nipples | CFU/mL |
| | | Estimated | 18.3 lb./100 lb. × |
| | | Amount of | 946 mL* = 173 mL |
| | | Water (mL) | |
| | | Each Pig | |
| | | Would | |
| | | Consume | |
| | | CFU/Pig Dose | 3.50 × 10ˆ 5 |
| | | As Actually | CFU/mL × 173 |
| | | Administered | mL/pig dose |
| | | | =6.06 × 10ˆ 7 |
| | | | CFU/pig dose |
| Second | 0 | 1.63E+05 | 1.26E+05 |
| Second | 1 | 1.02E+05 | 1.08E+05 |
| Second | 2 | 1.31E+05 | 1.35E+05 |
| Second | 3 | 1.59E+05 | 1.59E+05 |
| Second | 4 | 1.88E+05 | 1.80E+05 |
| Second | 5 | 1.54E+05 | 1.51E+05 |
| Second | 6 | 1.43E+05 | 8.50E+04 |
| Second | Average | 1.49E+05 | 1.35E+05 |
| | | Average of | 1.42 × 10ˆ 5 |
| | | Two Nipples | CFU/mL |
| | | Estimated | 35.5 lb./100 lb. × |
| | | Amount of | 946 mL* = 336 mL |
| | | Water (mL) | |
| | | Each Pig | |
| | | Would | |
| | | Consume | |
| | | CFU/Pig Dose | 1.42 × 10ˆ 5 |
| | | As Actually | CFU/mL × 336 |
| | | Administered | mL/pig dose |
| | | | =4.77 × 10ˆ 7 |
| | | | CFU/Pig Dose |

*946 mL is based on the calculation that a 100 lb pig would drink 1 gallon (3785.4 mL) of water during 24 hours, therefore, a 100 lb. pig would drink 946 mL of water during 6 hour vaccination period.

Comparison of Viable Count of Vaccine Organism Between Stock Solution and Nipple Samples

The viable count of vaccine organism between the stock solution and nipple samples was compared. The results at first and second vaccination are shown in Table 1 and Table 2, respectively. At first vaccination, the average viable count of the stock solution was 1.36×10⁸ CFU/mL. The average CFU/mL of the two nipples was 3.49×10⁵ CFU/mL and the average theoretical CFU/mL (average CFU/mL of stock solution/128) was 1.06×10⁶ CFU/mL. The difference between the average of nipples and theoretical concentration was 0.48 log value. Similarly, at second vaccination, the average viable count of the stock solution was 3.51×10⁷ CFU/mL. The average CFU/mL of the two nipples was 1.42×10⁵ CFU/mL and the average theoretical CFU/mL (average CFU/mL of stock solution/128) was 2.74×10⁵ CFU/mL. The difference between the average of nipples and theoretical concentration was 0.29 log value. Data collected from this study indicate that the average delivery concentration between nipple samples and stock solution was not far from the expectation (i. e. less than 0.5 log) and falls within normal range expected for CFU determination.

TABLE 1
First Vaccination: Comparison of Viable Count of Vaccine
Organism Between Stock Solution and Nipple Samples

| | Stock | | | Average of | | Difference Between the Average |
|---|---|---|---|---|---|---|
| Sample Time | Solution | Nipple 1 | Nipple 2 | two Nipples | Theoretical* | of the Nipples and Theoretical |
| Hour | (CFU/mL) | (CFU/mL) | (CFU/mL) | (CFU/mL) | CFU/mL | CFU/mL (log value) |
| 0 | 2.70E+08 | 2.81E+05 | 2.68E+05 | 2.75E+05 | 2.11E+06 | −0.886 |
| 1 | 1.11E+08 | 3.86E+05 | 2.86E+05 | 3.36E+05 | 8.67E+05 | −0.412 |
| 2 | 9.12E+07 | 2.91E+05 | 3.48E+05 | 3.20E+05 | 7.13E+05 | −0.348 |
| 3 | 9.48E+07 | 3.57E+05 | 3.71E+05 | 3.64E+05 | 7.41E+05 | −0.308 |
| 4 | 2.02E+08 | 4.45E+05 | 4.24E+05 | 4.35E+05 | 1.58E+06 | −0.560 |
| 5 | 9.48E+07 | 4.73E+05 | 4.34E+05 | 4.54E+05 | 7.41E+05 | −0.213 |
| 6 | 9.04E+07 | 2.74E+05 | 2.54E+05 | 2.64E+05 | 7.06E+05 | −0.427 |
| Average | 1.36E+08 | 3.58E+05 | 3.41E+05 | 3.49E+05 | 1.06E+06 | −0.484 |

The proportioner was adjusted at 1:128 delivery ratio.
*Theoretical CFU/mL was calculated based on stock solution/128.

TABLE 2
Second Vaccination: Comparison of Viable Count of Vaccine
Organism Between Stock Solution and Nipple Samples

| | Stock | | | Average of | | Difference Between the Average |
|---|---|---|---|---|---|---|
| Sample Time | Solution | Nipple 1 | Nipple 2 | two Nipples | Theoretical* | of the Nipples and Theoretical |
| Hour | (CFU/mL) | (CFU/mL) | (CFU/mL) | (CFU/mL) | CFU/mL | CFU/mL (log value) |
| 0 | 3.21E+07 | 1.63E+05 | 1.26E+05 | 1.45E+05 | 2.51E+05 | −0.239 |
| 1 | 3.53E+07 | 1.02E+05 | 1.08E+05 | 1.05E+05 | 2.76E+05 | −0.419 |
| 2 | 3.44E+07 | 1.31E+05 | 1.35E+05 | 1.33E+05 | 2.69E+05 | −0.305 |
| 3 | 3.65E+07 | 1.59E+05 | 1.59E+05 | 1.59E+05 | 2.85E+05 | −0.254 |
| 4 | 3.66E+07 | 1.88E+05 | 1.80E+05 | 1.84E+05 | 2.86E+05 | −0.191 |
| 5 | 3.63E+07 | 1.54E+05 | 1.51E+05 | 1.53E+05 | 2.84E+05 | −0.269 |
| 6 | 3.42E+07 | 1.43E+05 | 8.50E+04 | 1.14E+05 | 2.67E+05 | −0.370 |
| Average | 3.51E+07 | 1.49E+05 | 1.35E+05 | 1.42E+05 | 2.74E+05 | −0.286 |

The proportioner was adjusted at 1:128 delivery ratio.
*Theoretical CFU/mL was calculated based on stock solution/128.

Observation Post Each Vaccination

The vaccinated pigs were observed for clinical signs associated with erysipelas through eight days post each vaccination to ensure safety of the vaccine. Daily rectal temperatures were also taken during the observation period.

Observation and Challenge Procedures

Three weeks post second vaccination, all pigs from both vaccinate and control groups were challenged with a virulent strain of E. rhusiopathiae. The challenge strain (E1-6P, IRP ERC Serial 4, USDA, APHIS, CVB-L, 9-97 challenge) was prepared as described in SOP # a11-015-02 (E. rhusiopathiae Serotype 1, Challenge for SPF Swine). Briefly, the culture was received from CVB-L, Ames, Iowa, and grown in modified Feist medium. The CFU/mL was determined and then the culture was frozen for storage. For challenge, the frozen stock was quick-thawed and each pig received one mL of the challenge culture intramuscularly in the neck area. The challenge dose (5.7×10⁴ CFU/mL) was confirmed by CFU counts of the challenge material on TSA II blood agar plates prior to and after challenge. All pigs were observed for clinical signs associated with erysipelas and the rectal temperatures were measured for two days prior to and for seven days post challenge in accordance with 9 CFR 113.67.

A detailed protocol for carrying out the challenge experiment is provided below:

A. Materials

1. Protective Equipment (gloves, coat, and safety glasses).

2. One vial, E. rhu. Strain E1-6P IRP ERC Serial 4-9/97, first passage from NVSL challenge culture.

3. Sterile Tryptic soy broth.

4. Susceptible pigs from an SPF herd.

5. Syringes.

6. Needles.

7. Rectal thermometer.

8. Sterile pipettes.

9. Sterile dilution tubes.

10. Blood agar plates.

11. Sterile inoculation loops.

12. 200 ul pipettor.

13. Sterile pipette tips.

B. Methods

1. Don protective clothing and accessories (gloves, coat, and safety glasses) to protect caretaker from potential hazards. Erysipelothrix rhusiopathiae is a known human pathogen that may cause septicemia, skin lesions, arthritis, and/or death. It is transmitted through body fluids and open sores. Any suspected exposure should be reported immediately.

2. On days −2, −1 and 0 prior to challenge, take a rectal temperature (this serves as the baseline temperature for each pig). Record the temperatures.

3. Aseptically, prepare the challenge material (E. rhu. Strain E1-6P IRP ERC Serial 4-9/97) just prior to its administration. Quick thaw the vial of challenge by rubbing it in your hands. Record the time the seed is thawed on Attachment II. Shake the seed vial lightly, and dilute it in Trypticase Soy Broth (TSB) to a final concentration of 6.5×10⁴ CFU/ml using the following method (the seed concentration is approximately 2.15×10⁷ CFU/ml). Aseptically, add 0.5 ml of the challenge seed material to 4.5 ml of sterile TSB (Tube 1-2.15×10⁶ CFU/ml). Hold tube 1 at room temperature for 15 minutes, then thoroughly mix tube 1 and aseptically add 3.0 ml of tube 1 to 7.0 ml of sterile TSB (Tube 2-6.5×10⁵ CFU/ml). Thoroughly mix tube 2 and aseptically make a 1:10 dilution of tube 2 in TSB (Tube 3-6.5×10⁴ CFU/ml). Make enough of this dilution to challenge the appropriate number of pigs. (i.e. If you need to challenge 25 pigs with a 1.0 ml dose of 6.5×10⁴ CFU/ml challenge material, make at least 30 ml of 6.5×10⁴ CFU/ml challenge material. To do this, aseptically add 3.0 ml of tube 2 to 27.0 ml sterile TSB.) Keep all challenge material and dilution tubes on ice until the time of challenge.

4. Determine the concentration of the challenge material. Thoroughly mix tube 3 and aseptically add 0.5 ml of tube 3 to 4.5 ml of sterile TSB (Tube 4-6.5×10³ CFU/ml). Thoroughly mix tube 4 and aseptically add 0.5 ml of tube 4 to 7.0 ml of sterile TSB (Tube 5-4.3×10² CFU/ml).

5. Label 3 sheep blood agar (SBA) plates with “tube 5-prechallenge E. rhusiopathiae”, the date and initials. Thoroughly mix tube 5, and aseptically remove three separate 0.1 ml aliquots from tube 5 and place it on three SBA plates. Use a sterile inoculating loop to spread the samples over the surface of the SBA plates without getting too close to the edge. Incubate the plates 20-48 hours at 37□C. Record the time the prechallenge CFUs were plated. Put all dilution tubes on ice.

6. Challenge all pigs in the neck muscle with 1.0 ml, IM, of the challenge material from tube 3 (6.5×10⁴ CFU/ml) prepared in step IV.B.3. Record on which side of the neck the pigs were challenged. Keep all challenge material on ice during the challenge period.

7. After the pigs are challenged, thoroughly mix the contents of tube 5. Label three SBA plates with “tube 5-post challenge E. rhusiopathiae and the date. Aseptically, remove three separate 0.1 ml aliquots from tube 5 and place it on three sheep blood agar plates. Use a sterile inoculating loop to spread the samples over the surface of the SBA plates without getting to close to the edge. Incubate the plates 20-48 hours at 37□C. Record the time the post challenge CFUs were plated and calculate the time it took from the time the challenge material was thawed until the post challenge CFUs were done.

8. Take and record the temperature of each pig for seven consecutive days. Check each pig for clinical signs of erysipelas (depression with anorexia, stiffness, and/or joint involvement, moribundity with or without metastatic skin lesions) and record any observations. Also, check and record any injection site reactions, generalized patchy dermal redness, inappetance, or cyanosis.

9. A veterinarian should perform a necropsy and determine the cause of death of each pig that dies during the study but has not shown clinical signs of erysipelas.

10. Dispose of any remaining challenge material by incineration or autoclaving.

11. Count and average the number of colonies on the duplicate plates and record.

C. Calculations/lnterpretations

1. A control pig is considered positive for Erysipelas if it has clinical signs and/or a temperature of □ 105.6□F. for two consecutive days (excluding prechallenge days). (See 9 CFR §113.67). Pigs meeting the criteria to be considered positive may be treated with penicillin to relieve pain and distress at the discretion of the site supervisor or attending veterinarian.

2. At least 80% of the control pigs must show positive signs of Erysipelas during the observation period for the challenge to be valid. (See 9 CFR §113.67).

3. Multiply the average number of colonies counted times the final dilution plated. Average the concentrations of the pre and post challenge CFU results. The average concentration of challenge material should be between 5×10⁴ and 9×10⁴ CFU/ml for a valid challenge.

Clinical Signs and Temperature Post First Vaccination

All vaccinated pigs were observed until eight days post first vaccination and none of the pigs showed any clinical signs associated with erysipelas. Most pigs had a normal temperature during the post vaccination observation period, except for two pigs which had a single day temperature of 104.6° F. on 4DPV1 and 5 DPV1, respectively. No clinical signs were observed in the above two pigs. Some of the vaccinated pigs showed a temperature at 1° F. above baseline temperature during the observation period, which may have resulted from exciting the pigs during handling. Likewise, some non-vaccinated controls (such as pigs also had single or two days high temperatures without any clinical signs.

Clinical Signs and Temperature Post Second Vaccination

None of the vaccinated pigs showed any clinical signs associated with erysipelas through eight days post second vaccination. All pigs had a normal temperature during the observation period, except for one pig which had a single day temperature of 104.2° F. on 6DPV2 and another pig which had a temperature of 104.1° F. on 5 and 6DPV2, respectively. Both of these pigs did not show any clinical signs during the observation period. Similarly, one control pig showed a single day temperature of 104.3° F. on 7DPV2 without any clinical signs. These single day high temperatures probably resulted from exciting the pigs during handling. Data collected from both clinical observations and temperatures post each vaccination demonstrate that this vaccine strain is safe for pigs and will not cause clinical signs associated with erysipelas after vaccination.

Clinical Observations Post Challenge

At twenty-one days post second vaccination, the twenty vaccinates and ten controls were challenged with a virulent strain of E. rhusiopathiae. All pigs were observed for clinical signs associated with erysipelas and rectal temperatures were measured for two days prior to and for seven days post challenge.

Clinical Signs of Control Pigs Post Challenge

All non-vaccinated controls (100%) developed severe clinical signs associated with erysipelas, including arthritis, generalized patchy redness (diamond-skin lesions), lethargy, anorexia, depression and sudden death. At four days post challenge four control pigs, O404, O417, O421 and O432 were dead. Pigs O406 and R73 were found dead on 5DPC and pig O403 was dead on 6DPC. At seven days post challenge seven out of ten (70%) of the control pigs were dead. Pig O403 had a temperature of 105.7° F. on 5DPC before death. Pig O404 and O406 had temperatures of 103.1° F. and 102.4° F., respectively, before death. Pigs O417, O421, O432 and R73 had temperatures at 105.2° F., 104.9° F., 99.5° F. and 105.6° F., respectively before death. Three control pigs, O411, O426 and O429 survived challenge with severe clinical signs.

Clinical Signs of Vaccinated Pigs Post Challenge

One hundred percent (20 out of 20) of the vaccinates did not show typical clinical signs related to erysipelas during the observation period. Pig O409 showed injection site redness at 2DPC. None of the vaccinated pigs showed temperature above 104.0° F. during observation period post challenge. Data collected from the vaccinated pigs demonstrated that 100% of the vaccinates were protected from E. rhusiopathiae challenge. These results satisfactorily meet the 9 CFR requirements to qualify an efficacious vaccine to protect pigs from E. rhusiopathiae infection.

i E. rhusiopathiae Isolation from Pigs Post Challenge

E. rhusiopathiae isolation was conducted from the blood, spleen, liver and mesenteric lymph node collected from the control pigs post challenge or at necropsy. As observed, E. rhusiopathiae was isolated from samples collected from control pigs O403, O406, O411, O426, O429 and R73. Pigs O404, O417, O421 and O432 were found dead on 4DPC and no samples were collected at that time. Blood samples were also collected from vaccinated pigs at 7 DPC and no E. rhusiopathiae was isolated from the vaccinated pigs. Results of E. rhusiopathiae isolation from control pigs meet the 9 CFR requirements for a valid E. rhusiopathiae challenge.

Conclusion

Data from this study demonstrate that a flavored vaccine formulation of the invention, in this case, comprising Erysipelothrix Rhusiopathiae Vaccine, Avirulent Live Culture, mass administered, according to the method of the invention, at the rate of approximately 6.06×10⁷ CFU/dose through the drinking water using an automated water proportioning device, is safe and efficacious to protect pigs from disease caused by E. rhusiopathiae infection. Results from this study satisfactorily meet the requirements stated in 9 CFR 113.67 and qualify Erysipelothrix Rhusiopathiae Vaccine, Avirulent Live Culture, for licensure.

### EXAMPLE 2

Orally Administered Flavored Vaccine Compared to Unflavored

In order to demonstrate that the flavored orally administered vaccine of the invention provided greater protection against infection as compared to unflavored, a vaccination protocol similar to the one described in Example 1 was carried out utilizing a strawberry flavored vaccine formulation with lyophilized Erysipelothrix rhusiopathiae as antigen, an unflavored vaccine formulation with lyophilized Erysipelothrix rhusiopathiae as antigen, and a control formulation with no flavorant or antigen added. All vaccine and control formulations were prepared as described in Example 1. Challenge Experiments were carried pout as described in Example 1.

The experiments and data are described in the tables below:

TABLE 4
Administration of Flavored Vaccine Formulation - Study I

| | | | % Protection Upon |
|---|---|---|---|
| Group | Dose Per Pig | Number of Pigs | Challenge |
| 1 | Single Dose 5 × 10⁷ | 5 | 100% |
| 2 | Single Dose 5 × 10⁸ | 5 | 100% |
| 3 | Single Dose 5 × 10⁷ | 5 | 100% |
| 4 | Single Dose 5 × 10⁸ | 5 | 100% |
| Control | NA | 8 | NA-100% Disease |

TABLE 5
Administration of Flavored Vaccine Formulation - Study II

| | | | % Protection Upon |
|---|---|---|---|
| Group | Dose Per Pig | Number of Pigs | Challenge |
| Vaccinate | Single Dose 1 × 10⁷ | 20 | 50% |
| Control | NA | 10 | NA-100% Disease |
| Vaccinate | 2 Doses 1 × 10⁷/dose | 20 | 75% |
| Control | NA | 10 | NA-100% Disease |

TABLE 6
Administration of Unflavored Vaccine Formulation

| | | | % Protection Upon |
|---|---|---|---|
| Group | Dose Per Pig | Number of Pigs | Challenge |
| 1 | Single Dose 1 × 10⁷ | 21 | 10% |
| 2 | Single Dose 2 × 10⁷ | 18 | 22% |
| Control | NA | 10 | NA-100% Disease |

### EXAMPLE 3

In order to demonstrate that the antigen is active in the vaccine formulations without flavoring, pigs were administered a single dose of vaccine formulated without flavoring by syringe. These data are provided in Table 7 below and demonstrate that the antigen is active and provides evidence that the flavorant provides for a greater intake by the pigs of the flavored orally administered vaccine in the drinking water.

TABLE 7
Syringe Delivery of Unflavored Vaccine

| | | | % Protection Upon |
|---|---|---|---|
| Group | Dose Per Pig | Number of Pigs | Challenge |
| Vaccinate | Single Dose 1 × 10⁷ | 3 | 100% |
| Control | NA | 3 | NA-100% Disease |

Reference

M. L. Augenstein, L. J. Johnston, G. C. Shurson, J. D. Hawton and J. E. Pettigrew. Formulating Farm-Specific Swine Diets; University of Minnesota Extension Service. 1994.

1. A method of providing protection against disease in an animal comprising:
(a) admixing a water soluble palatable flavorant with a water soluble vehicle for administration of an orally administered vaccine;
(b) further admixing with the mixture of step (a), an antigen selected from the group consisting of a bacterium and a virus as an active component of the orally administered vaccine; and
(c) administering the orally administered vaccine of step (b) to an animal to provide protection against disease associated with infection by the antigen.

2. The method of claim 1, wherein the antigen is capable of causing disease in an animal selected from the group consisting of swine, poultry, cattle, sheep, goats, horse, cat and dog.

3. The method of claim 2, wherein the antigen is selected from the group consisting of Erysipelothrix rhusiopathiae, Actinobacillus pleuroneumonla, Mycoplasma hyopneumonlae, E. coli K88, K99, F41 and 987P, Clostridium perferingens type c, Salmonella choleraesuls, Pasterurella muitocida, Bordetella bronchiseptica, Leptospira bratislava, Leptospira canicola, Leptospira grippotyphosa, Leptospira hardjo, Leptospira promona, Leptospira ictero, Porcine Influenza virus, Circovirus, PRRS virus, Swine pox, Rotavirus, Porcine Respiratory Coronavirus, Parvo virus, Pseudorabies, transmissible gastroenteritis agent, Streptococcus equi, Clostridium tetanus, Equine Influenza Virus A1 and A2 strains, Equine Rhinopneumonids type 1, 1b and 4, Eastern Equine Encephalomyelitis, Western Equine Encephalomyelitis, Venezuelan Equine Encephalomyelitis, Equine Rotavirus, E. coli O157:H7, Pasterurella multocida, Pasterurella haemolytica, Clostridium perferingens type D, Clostridium chauvoel, Clostridium novyl, Clostridium septicum, Clostridium haemolyticum, Clostridium sodellii, Salmonella dublin, Salmonella typhimurium, Bovine Rotavirus, Bovine coronavirus, Bovine rhinotracheitis, Bovine diarrhea virus, Parainfluenza-3, Respiratory syncytial virus, Sepullina pilosicoli, Marek's disease virus, Infectious bursal disease, Infectious bronchitis, Newcastle disease virus, Reo virus, Turkey rhinotracheltis, Couidiosis, Canine Borrella burgdorferi, Canine Ehrlichia canis, Canine Bordetella bronchiseptica, Canine Giardia lamblia, Canine distemper, Canine Adenovirus, Canine Coronavirus, Canine Parainfluenza, Canine Parvovirus, Canine Rabies, Feline Chlamydia psittaci, Feline immunodeficiency virus, Feline infectious peritonitis virus, Feline leukemia virus, Feline rhinotrachelitis, Feline Panleukopenia, Feline rabies.

4. The method of claim 1, wherein the vaccine is administered through drinking water.

5. The method of claim 1, wherein the animal is selected from the group consisting of swine, poultry, cattle, sheep, goats, horse, cat and dog.

6. The method of claim 1, wherein the animal is selected from the group consisting of swine and poultry.

7. The method of claim 6, wherein the administration of the orally administered vaccine is a mass administration through drinking water.

8. The method of claim 7, wherein the animal is a pig and the antigen is Erysipelothrix rhusiopathiae.

9. The method of claim 1, wherein the animal is selected from the group consisting of dog and cat.

10. The method of claim 7, wherein the administration of the orally administered vaccine into the mouth through a syringe.

11. A method of inducing increased intake of an orally administered vaccine by an animal comprising:
(a) admixing a water soluble palatable flavorant with a water soluble vehicle for administration of an orally administered vaccine;
(b) further admixing with the mixture of step (a), an antigen selected from the group consisting of a bacterium and a virus as an active component of the orally administered vaccine; and
(c) administering the vaccine admixture of step (b) orally to the animal;
(d) inducing the increased intake of the orally administered vaccine with the flavorant.

12. The method of claim 11, wherein the antigen is capable of causing disease in an animal selected from the group consisting of swine, poultry, cattle, sheep, goats, horse, cat and dog.

13. The method of claim 12, wherein the antigen is selected from the group consisting of Erysipelothrix rhusiopathiae, Actinobacillus pleuroneumonla, Mycoplasma hyopneumonlae, E. coli K88, K99, F41 and 987P, Clostridium perferingens type c, Salmonella choleraesuls, Pasterurella muitocida, Bordetella bronchiseptica, Leptospira bratislava, Leptospira canicola, Leptospira grippotyphosa, Leptospira hardjo, Leptospira promona, Leptospira ictero, Porcine Influenza virus, Circovirus, PRRS virus, Swine pox, Rotavirus, Porcine Respiratory Coronavirus, Parvo virus, Pseudorabies, transmissible gastroenteritis agent, Streptococcus equi, Clostridium tetanus, Equine Influenza Virus A1 and A2 strains, Equine Rhinopneumonids type 1,1b and 4, Eastern Equine Encephalomyelitis, Western Equine Encephalomyelitis, Venezuelan Equine Encephalomyelitis, Equine Rotavirus, E. coli O157:H7, Pasterurella multocida, Pasterurella haemolytica, Clostridium perferingens type D, Clostridium chauvoel, Clostridium novyl, Clostridium septicum, Clostridium haemolyticum, Clostridium sodellii, Salmonella dublin, Salmonella typhimurium, Bovine Rotavirus, Bovine coronavirus, Bovine rhinotracheitis, Bovine diarrhea virus, Parainfluenza-3, Respiratory syncytial virus, Sepullina pilosicoli, Marek's disease virus, Infectious bursal disease, Infectious bronchitis, Newcastle disease virus, Reo virus, Turkey rhinotracheltis, Couidiosis, Canine Borrella burgdorferi, Canine Ehrlichia canis, Canine Bordetella bronchiseptica, Canine Giardia lamblia, Canine distemper, Canine Adenovirus, Canine Coronavirus, Canine Parainfluenza, Canine Parvovirus, Canine Rabies, Feline Chlamydia psittaci, Feline immunodeficiency virus, Feline infectious peritonitis virus, Feline leukemia virus, Feline rhinotrachelitis, Feline Panleukopenia, Feline rabies.

14. The method of claim 11, wherein the vaccine is administered through drinking water.

15. The method of claim 11, wherein the animal is selected from the group consisting of swine, poultry, cattle, sheep, goats, horse, cat and dog.

16. The method of claim 15, wherein the animal is selected from the group consisting of swine and poultry.

17. The method of claim 16, wherein the administration of the orally administered vaccine is a mass administration through drinking water.

18. The method of claim 17, wherein the animal is swine and the antigen is Erysipelothrix rhusiopathiae.

19. The method of claim 11, wherein the animal is selected from the group consisting of dog and cat.

20. The method of claim 19, wherein the administration of the orally administered vaccine is at the back into the mouth through a syringe.

21. An orally administered animal vaccine formulation comprising as an active component an antigen selected from the group consisting of a bacterium and a virus, a water soluble palatable flavorant and a water soluble vehicle for administration of the orally administered animal vaccine.

22. The vaccine formulation of claim 21, wherein the antigen is capable of causing disease in an animal selected from the group consisting of swine, poultry, cattle, sheep, goats, horse, cat and dog.

23. The vaccine formulation of claim 22, wherein the antigen is selected from the group consisting of Erysipelothrix rhusiopathiae, Actinobacillus pleuroneumonla, Mycoplasma hyopneumonlae, E. coli K88, K99, F41 and 987P, Clostridium perferingens type c, Salmonella choleraesuls, Pasterurella muitocida, Bordetella bronchiseptica, Leptospira bratislava, Leptospira canicola, Leptospira grippotyphosa, Leptospira hardjo, Leptospira promona, Leptospira ictero, Porcine Influenza virus, Circovirus, PRRS virus, Swine pox, Rotavirus, Porcine Respiratory Coronavirus, Parvo virus, Pseudorabies, transmissible gastroenteritis agent, Streptococcus equi, Clostridium tetanus, Equine Influenza Virus A1 and A2 strains, Equine Rhinopneumonids type 1, 1b and 4, Eastern Equine Encephalomyelitis, Western Equine Encephalomyelitis, Venezuelan Equine Encephalomyelitis, Equine Rotavirus, E. coli O157:H7, Pasterurella multocida, Pasterurella haemolytica, Clostridium perferingens type D, Clostridium chauvoel, Clostridium novyl, Clostridium septicum, Clostridium haemolyticum, Clostridium sodellii, Salmonella dublin, Salmonella typhimurium, Bovine Rotavirus, Bovine coronavirus, Bovine rhinotracheitis, Bovine diarrhea virus, Parainfluenza-3, Respiratory syncytial virus, Sepullina pilosicoli, Marek's disease virus, Infectious bursal disease, Infectious bronchitis, Newcastle disease virus, Reo virus, Turkey rhinotracheltis, Couidiosis, Canine Borrella burgdorferi, Canine Ehrlichia canis, Canine Bordetella bronchiseptica, Canine Giardia lamblia, Canine distemper, Canine Adenovirus, Canine Coronavirus, Canine Parainfluenza, Canine Parvovirus, Canine Rabies, Feline Chlamydia psittaci, Feline immunodeficiency virus, Feline infectious peritonitis virus, Feline leukemia virus, Feline rhinotrachelitis, Feline Panleukopenia, Feline rabies.

24. The vaccine formulation of claim 21, wherein the vehicle for administration is drinking water.

25. The vaccine formulation of claim 21, wherein the animal is a swine and the antigen is Erysipelothrix rhusiopathiae.

26. The vaccine formulation of claim 21, wherein the animal is selected from the group consisting of a dog and a cat and the vehicle for administration is a syrup.
